(19) United States
(12) Patent Application Publication
(10) Pub. No.: US 2010/0297056 A1
(43) Pub. Date: Nov. 25, 2010

(54) COMPOSITION BASED ON PERHYDROSQUALENE AND COLLAGEN-POLYVINYLPYRROLIDONE FOR FILLING MINOR CUTANEOUS DEPRESSIONS

(75) Inventors: Nantzin Martinez Fleischer De Leal, Mexico D.F (MX); Fernando Edgar Krotzsch Gomez, Mexico D.F (MX); Rosa Maria Salgado Curiel, Mexico D.F (MX)

Correspondence Address: BROOKS KUSHMAN P.C., 1000 TOWN CENTER, TWENTY-SECOND FLOOR, SOUTHFIELD, MI, 48075, US

(73) Assignee: ASPID, S.A. DE C.V., Colonia Portales, C.P., Mexico D.F. (MX)

(21) Appl. No.: 12/672,938

(22) PCT Filed: Aug. 11, 2008
(86) PCT No.: PCT/MX08/00106

§ 371 Date: Apr. 14, 2010

(30) Foreign Application Priority Data
Aug. 10, 2007 (MX) MX/A/2007/009730

(51) Int. Cl. A61K 31/79; A61Q 19/00

(52) U.S. Cl. 424/78.3

(57) ABSTRACT

The present invention concerns a composition based on collagen-polyvinylpyrrolidone and perhydrosqualene for filling by intracutaneous delivery route minor depressions of the skin in order to enhance the appearance thereof without causing a negative immune reaction or rejection of the extraneous body, a method for preparing the composition and the use thereof in the prevention and treatment of minor depressions in the skin by means of a temporary mechanical effect thereby avoiding the encapsulation of the substance, since depending among various factors on time, some compositions eventually cause a reaction to the extraneous body, leading to inflammation of the treated area and fibrosis. The composition as per the invention has the characteristic of being prepared to a controlled alkaline PH and forms a stable emulsion without foam.

### TECHNICAL FIELD

The instant invention relates to a composition based on collagen-polyvinylpyrrolidone and perhydrosqualene for filling minor cutaneous depressions through intra-cutaneous route to improve skin aspect that does not cause a negative immune reaction, including the rejection of extraneous bodies, a process to produce it, and its use in the treatment of minor skin depressions.

### BACKGROUND OF THE INVENTION

The physical appearance of people is an important factor for social adaptation; however, the environmental conditions, scars and their consequences, as well as the aging process alter the physiognomy and it becomes thus necessary to correct the undesirable modifications, such as depressions, marking the cutaneous topography, so that people can better adapt and develop in their social environments.

For this purpose several implant materials have been developed the object of which is to serve as intradermal support. Moreover, they can stimulate various metabolic mechanisms favoring the texture and consistency of the skin, both in the infiltrated site as well as in neighboring zones. Thus, the use of implant materials to fill minor cutaneous depressions is very important in health areas such as plastic surgery, reconstructive surgery, aesthetic medicine and dermatology, because they not only improve the tissue appearance but also favor the functionality of the neighboring structures. Notwithstanding the need to fill, it is important to consider that all “extraneous” materials intradermally infiltrated have the capacity to generate local reactions from simple inflammation to necrosis, either through ischemia or through adverse reaction to the extraneous body. Thus, the materials for filling minor cutaneous depressions must be inert, manageable, durable in the implanted site and, most important, easily accepted by the receiving body. It is important to state that it is impossible to have an absolutely inert material and thus a composition that can be easily and rapidly absorbed is sought in order to avoid its encapsulation and to reach effects that are merely temporary, although for extended periods of time. It is worth mentioning that having a temporary effect also favors a constant morphology of the user, because once the implant material is absorbed, the expression lines are formed again in the treated zone, contrary to the case of the definite filling materials, where upon occupying the space of the cutaneous defect, the natural gesticulation of the user will form new folds in the collateral areas, producing a modification in the morphology of the person in the middle term.

Some of the filling materials most commonly used are:

a) Metallic filaments placed in the cutaneous depression (gold filaments or threads).
b) Polymers composed of molecular chains or subunits, sometimes repeated, wherein the subunit structure and the cross linking among them determine the physical properties of the material (polysiloxane or silicone, polylactic acid, polyethylene, etc.) (Rubin J. P. et al., 1997, Sclafani A. P. et al., 1997).
c) The polyamides that are a group of polymerized amides (Rubin J. P. et al., 1997).
d) The autologous fat, obtained from sites that are rich in lipids such as the abdomen (Rasmussen J. E. et al., 1988).
e) Fibrin foam mixed with E-aminocaproic acid (Millikan L. et al., 1987).
f) Stabilized hyaluronic acid (Olenius M., 1998).
g) Dispersed fibrilar collagen (Tromovitch T. et al., 1984).
h) Dextran spheres positively charged and mixed with polyoxyethylene-sorbitan monooleate (Eppley B. et al., 1994).

In all these cases, except with regard to the first one, the excess correction of the defect is necessary through the administration of an excess of material, in order to compensate the part of the material that will be removed during the first days through local inflammation, said excess can range from 10 to 50% of the material to be implanted.

Now then, from the various materials mentioned, the object of some of them is simply to support internally the defect and said materials are denominated filling implants, while the object of the others is to stimulate a controlled inflammatory reaction favoring the synthesis and deposit of extracellular matrix (collagen, fibronectin, glycosaminoglycans, etc.) to correct the cutaneous depression with the material of the person itself; they are denominated filling forming substances. The above automatically classifies the various implant materials in such a way that both the user as well as the medical doctor can decide the type they want and the possible consequences of its administration, because even though it has not been possible yet to fully control the tissue reaction and there are collateral adverse reactions such as the formation of micro-calcifications, hyper-pigmentation, material encapsulation, transitory inflammation and even hypersensitivity of the components to the formula (Sheldon V. et al., 1990). On the other hand, the polysiloxane or low molecular weight silicone behaves mostly as an inert material that is not encapsulated and thus, it does not generate any type of collateral defect, and its application offers great advantages compared to other synthetic materials (Rubin J. P. et al., 1997).

Thus, in the search for safer materials with adequate retention in the implantation site, the perhydrosqualene was taken into consideration because it is a saturated hydrocarbon of animal origin that, adequately formulated, can behave as an inert material. Owing to its great purity, the perhydrosqualene is an odorless and limpid fluid oil that can adapt to the pharmaceutical preparations because of its characteristics, showing great stability because of its lack of susceptibility to oxidation of any type, and thus does not present rancidity risk.

From the dermatological point of view and taking into account its topical administration, the perhydrosqualene shows great affinity for the skin because it is a notable a touch sensation emollient. Moreover, the perhydrosqualene incorporates easily in the skin surface without leaving a greasy sensation, conferring at the same time a soft and silky aspect, representing moreover a privileged transfer vehicle of the cosmetic active ingredients to the absorbing surface of the follicular wall, improving thus its penetration (Flesh P., 1956-1957) and its efficacy increases because it is miscible with intercellular cement (Flesh P., 1962). Because of its compatibility with oils and lipophilic substances, perhydrosqualene emulsifies easily, conferring a fine, attractive and brilliant aspect to the formulation.

Based on the above and given its proven thermal stability, perhydrosqualene is used in the manufacture of skin lubricants, as suppository ingredient and as carrier for various dermatological active ingredients.

It is an object of the instant invention to offer a chemical composition applicable in medicine based on perhydrosqualene plus collagen-polyvinylpyrrolidone for filling minor cutaneous depressions.
It is thus an object of the instant invention to favor the continuity of the cutaneous relief when minor skin depressions exist.
It is another object of the instant invention to offer a dermal implantable composition based on perhydrosqualene for the temporary filling the cutaneous depressions.
It is another object of the instant invention to offer a dermal implantable composition based on collagen-polyvinylpyrrolidone that prevents the physiological encapsulation of perhydrosqualene.
It is another object of the instant invention to offer a dermal implantable chemical composition for the temporary filling of cutaneous depressions and that does not cause a severe inflammatory reaction that could lead to the physiological rejection of the administered material. Once the perhydrosqualene is accepted by the tissue it is not considered equal to the case when the perhydrosqualene is physico-chemically combined with collagen-polyvinylpyrrolidone.
Another object of the instant invention is to offer a dermal implantable chemical composition for the temporary filling of cutaneous depressions through the combination of collagen-polyvinylpyrrolidone with perhydrosqualene that favors the acceptation of the lipophilic material by the treated tissue, because of the immunomodulating properties of the collagen-polyvinylpyrrolidone.
It is another object of the instant invention to offer a dermal implantable composition for the temporary filling of the cutaneous depressions that advantageously competes against other non-combined collagen compositions, synthetic polymers or biological hydrocarbons.
It is another object of the instant invention to offer a composition based on collagen-polyvinylpyrrolidone and perhydrosqualene for filling through intracutaneous route minor skin depressions.
It is another object of the instant invention to improve the aspect of the skin of a user requiring it.

As a consequence, the field of action of the invention is determined in the area of chemical products applied to medicine.

### DETAILED DESCRIPTION OF THE INVENTION

The intradermal administration of non-reactive hydrophobic substances permits to lift the cutaneous surface through a mechanical effect. The effect will remain while the infiltrated material stays in the zone, which shall be temporary to prevent the encapsulation of the substance and the collateral folding of the treated zone. Even in this case, some compositions can generate a reaction against an extraneous body, deriving in the inflammation of the infiltrated region and fibrosis. For this reason, in the instant invention we have combined perhydrosqualene (2,6,10,15,19,23-hexamethyltetracosane) with collagen-polyvinylpyrrolidone, because this latter has inflammation modulating properties and can prevent the encapsulation derived from the localized fibrosis (patent 214259 Mexico, Krötzsch-Gómez FE, Furuzawa-Carballeda J, Reyes-Márquez R, Quiróz-Hernández E and Díaz de Leóon L. Cytokine expression is downregulated by collagen-polyvinylpyrrolidone in hypertrophic scars. J Invest Dermatol, 1998; 111:828-834. Cervanmtes-Sánchez C R., Olaya E, Testas M, García López N, I Coste G, Arrellín G, Luna A, Krótzsch E. Collagen-PVP a collagen synthesis modulator decreases intra-peritoneal adhesions. J Surg Res 2003; 110:207-210. Furuzawa-Carballeda J, Krótzsch E, Espinosa-Morales R, Alcalá M, Barile-Fabris L. Subcutaneous administration of collagen-polyvinylpyrrolidone down-regulates IL-1β, TNF-α, TGF-β1, ELAM 1 and VCAM-1 expression in scleroderma skin lesions. Clinical and Experimental Dermatology. 2005; 30:83-86).

The preparation of the chemical composition comprises 3 phases:

### 1. The Formation of the Collagen-Polyvinylpyrrolidone Complex.

The collagen-polyvinylpyrrolidone is a biological-synthetic copolymer involving gamma irradiation of the mixture of collagen-polyvinylpyrrolidone and polyvinylpyrrolidone in a slightly acidic pH (patent 214259, Mexico).

### 2. Alkalinization of the Collagen-Polyvinylpyrrolidone Copolymer.

The alkalinization is conducted until reaching a pH ranging from 9.0 to 10.5 that favors the integration of the other components when they are added. Moreover, crossing the isoelectric point of the collagen-polyvinylpyrrolidone to alkalinity (conferred essentially by the protein), a chemical composition is generated that has a minor capacity of generating pain in the application site compared to the case when the pH is acidic.

### 3. Formation of the Final Emulsion.

The alkaline copolymer of collagen-polyvinylpyrrolidone is mixed with the 2,6,10,15,19,23-hexamethyltetracosane at ratios ranging from 1:5.7 to 1:11.5, respectively.

Then, the mixture is emulsified through physical means that do not favor foam formation, until a stable product is obtained that does not separate through centrifugation or through moderate temperature changes.

Moreover, to the mixture of collagen-polyvinylpyrrolidone copolymer with the 2,6,10,15,19,23-hexamethyltetracosane, in the mentioned ratios, 0.1-0.3% dibutyl-lauroyl glutamide can be added, which is an aminoacid gelling agent, in order to generate a soft fluid gel that, upon being implanted, remains for longer period of time in the tissues where it has been deposited (Salgado Curiel Rosa María. Evaluación de un bioimplante cutáneo en un modelo murino: Estudio clínico e histológico. Escuela de Química. Universidad La Salle, 2001).

On the other hand, the mixture of collagen-polyvinylpyrrolidone copolymer and 2,6,10,15,19,23-hexamethyltetracosane can be enriched with polysiloxane form 200 to 350 cps (centipoises) in a ratio 1:3.8:2.5, respectively, in order to obtain a bioimplantation material that remains even longer in the administration zone, with the advantage that a part of it will remain without being metabolized and the aesthetic effect will have a larger impact (Salgado Curie Rosa María. Evaluación de un bioimplante cutáneo en un modelo murino: Estudio clínico e histológico. Escuela de Química. Universidad La Salle, 2001).

The collagen-polyvinylpyrrolidone and perhydrosqualene composition of the instant invention and/or its variations have the object of filling minor cutaneous depressions preventing an exaggerated reaction against the extraneous body, and thus the perhydrosqualene can modify the cutaneous relief temporarily and thus offer functional and aesthetic improvements to the tissues. The above statement derives from the preclinical and clinical tests conducted, wherein because of its complete stability, perhydrosqualene is inert and does not interfere with other materials employed in the primary packaging of the formula.

During the manufacturing process of the perhydrosqualene, from the standpoint of the perhydrosqualene purity, no organic solvents are employed and thus it is totally innocuous, which has been demonstrated through numerous toxicological studies establishing thus the mean lethal dose as: DL₅₀>20 ml/kg (Expedientes Toxicológicos, 1980). Another important aspect is the absence of cutaneous and ocular irritation in humans, as well as in subcutaneous injections in mice (CTFA 1957). It has also been established that the perhydrosqualene is a non-metabolizable hydrocarbon, and is thus excreted through natural routes (Squalane 1980).

In prior studies, it has been demonstrated that the interaction of vesicular structures formed by perhydrosqualene-water and trehalose dimycolate emulsion with the immunocompetent cells has been well accepted because of the improvement of the defense mechanisms presented by the host and because of the induction of a non-specific resistance against viruses, parasites, bacteria and some tumors. Studies have also been conducted that demonstrated that perhydrosqualene administered orally increases fecal excretion, reducing thus blood drug level.

Because of the biocompatibility presented by the perhydrosqualene, it has been reported that a group of rats receiving an oral treatment with this hydrocarbon during a three-month period did not show signs of toxicity; in the same way, a study was conducted with dogs that were administered perhydrosqualene at a dose of 1200 mg/kg during 14 days, observing perhydrosqualene excretion in a percentage of 65-90% per day while at blood level, it only presented 30 ppm. After the first dose, on day 56, perhydrosqualene was not detectable in blood, and thus the results suggest the mobilization and the continuous excretion of perhydrosqualene through feces and skin.

Because of the above, a murine model essay of intradermal administration was designed, wherein several implant materials were evaluated such as natural and synthetic jojoba oil, peanut oil and perhydrosqualene, mixed with collagen-polyvinylpyrrolidone, various emulsifiers and a gelling agent (dibutyl-lauroyl glutamide). The administration of the implant materials generated papules, formed by the deposition of the material in the reticular dermis of the back of the rats. The papules were evaluated with regard to the tolerance of the tissue to the implant through a visual analog scale during a period of about 30 days, with their intervals; moreover, the eventual occurrence of skin changes such as irritation, ischemia formation and in some cases tissue necrosis related to the implanted material were reported, as well as the durability of the bioimplant that was quantified through a sensorial analog scale derived from the papule palpation.

In the same way, histological rat skin changes were evaluated through biopsies obtained from the implant infiltrated site, said biopsies were collected at different times in order to determine whether changes occurred in the internal skin structure, as well as to observe the implant site, the inflammatory infiltrate, the ratio between type I and type III collagens, as well as the possible formation of granulomes.

The results obtained from this study showed that, in the case of reactivity and tolerance to the materials, both the natural and synthetic jojoba oil as well as peanut oil presented rat skin irritation during the first days, moreover ischemia and tissue necrosis were observed on the third and fourth day, respectively.

The formulations including perhydrosqualene and/or polysiloxane mixed with additives such as collagen-polyvinylpyrrolidone and/or gelling agent, dibutyl-lauroyl glutamide, showed total tolerance and acceptable durability in the tissue; and gave satisfactory results because no irritation, ischemia or necrosis was observed on the back of the rat several days after the infiltration. Moreover, the durability presented by the perhydrosqualene was constant even 15 days after its administration. With regard to the histological results, it can be mentioned that no inflammatory infiltrates affecting the skin structure were observed and, at the same time, type III collagen was progressively generated, resulting in a healthy skin. Because of the above, the formulation based on perhydrosqualene can be considered as possible implant material. (Salgado Curiel Rosa María. Evaluación de un bioimplante cutáneo en un modelo murino: Estudio clínico e histológico. Escuela de Química. Universidad La Salle, 2001).

Afterwards, a clinical assay was conducted to determine the tolerance, dose and re-application frequency of the prepared composition based on collagen-polyvinylpyrrolidone and perhydrosqualene. The results obtained are mentioned hereinafter.

The patient selection was conducted through a general health diagnosis, as well as based on a specific diagnosis of his facial cutaneous state. Moreover, tests of sensitivity to the components of the chemical composition were conducted through an intradermal reaction test evaluated during 48 hours.

Each patient on the exploration table received the appropriate hygiene measures and local anesthesia. The infiltration of collagen-polyvinylpyrrolidone/perhydrosqualene was conducted intradermally, taking care of the needle direction angle and following the trajectory of the cutaneous depression.

The technique used for cutaneous depression was linear tracking in simple layer, i.e. the material is progressively infiltrated, while the needle is progressively withdrawn from the application zone. In the case of periorbicular cutaneous depressions, the technique used was puncture, wherein the chemical composition was completely deposited in the application site.

The result obtained was the correction of the cutaneous depression through a temporary filling system with a variable duration ranging from 3 to 6 months. None of the patients studied showed either positive intradermal reactions or adverse collateral effects, even 12 months after the last chemical composition administration. No liver, renal o blood alterations were found.

Hereinafter the doses, frequencies and characteristics of the studied patients after the intradermal application of collagen-polyvinylpyrrolidone/perhydrosqualene are presented:

INFILTRATION OF COLLAGEN-
POLYVINYLPYRROLIDONE/PERHYDROSQUALENE IMPLANT

| | Number of | | |
|---|---|---|---|
| | patients | | |
| Region | (age) | Dose (ml) | Frequency |
| Forehead | 1₍₃₉₎ | 1 | 2 infiltrations, one every |
| | | | 30 days |
| Periorbicular | 1₍₄₃₎ | 1 | 1 infiltration every 4 |
| | | | months |
| Brow | 1₍₄₂₎ | 0.25 | 5 infiltrations, one every |
| | | | 30 days |
| Brow | 2₍₄₁₎ | 0.5 | 6 infiltrations, one every |
| | | | three months |
| Brow | 3₍₄₇₎ | 0.5 | 2 infiltrations every 15 |
| | | | days |
| Brow | 4₍₃₁₎ | 0.5 | 2 infiltrations every year |
| Brow | 5₍₄₃₎ | 0.25 | 2 infiltrations every 15 |
| | | | days |
| Nasogenian | 1₍₃₃₎ | 1 | Start |
| | | 1.5 | 1 infiltration every 4 |
| | | | months |
| | | 1 | 1 infiltration every 4 |
| | | | months |
| Nasogenian | 2₍₄₀₎ | 1 | 1 infiltration every 30 |
| | | | days |
| Nasogenian | 3₍₃₀₎ | 0.5 | 4 infiltrations, one every |
| | | | 15 days |
| | | 1 | 4 infiltrations, one every |
| | | | 15 days |
| | | 3.5 | 1 infiltration every 30 |
| | | | days |
| | | 0.5 | 5 infiltrations, one every |
| | | | 15 days |
| | | 1.2 | 2 infiltrations, one every |
| | | | 30 days |
| | | 0.5 | 2 infiltrations, one every |
| | | | 15 days |
| | | 1 | 2 infiltrations, one every |
| | | | 15 days |
| | | 0.5 | 2 infiltrations, one every |
| | | | 30 days |
| | | 1 | 8 infiltrations, one every |
| | | | 30 days |
| Nasogenian | 4₍₄₇₎ | 1 | 2 infiltrations, one every |
| | | | 30 days |
| Nasogenian | 5₍₃₀₎ | 3.5 | 3 infiltrations, one at the |
| | | | start, 1 at 19 months and 1 |
| | | | at 6 months |
| Nasogenian | 6₍₂₅₎ | 2.5 | 2 infiltrations, one every |
| | | | 30 days |
| Nasogenian | 7₍₂₈₎ | 1.5 | 2 infiltrations, one every |
| | | | 15 days |
| Lateral | 1₍₃₇₎ | 3 | Start |
| commissures | | 3.5 | 1 infiltration every 30 |
| | | | days |
| | | 2 | 2 infiltrations, one every |
| | | | 4 months |
| | | 3 | 1 infiltration every 3 |
| | | | months |
| Lateral | 2₍₈₀₎ | 4 | 2 infiltrations, one every |
| commissures | | | 9 months |
| Forehead and | 1₍₃₉₎ | 1.5 | 1 infiltration |
| nasogenian | | | |
| Forehead and | 2₍₄₅₎ | 2.5 | Start |
| nasogenian | | 2 | 1 infiltration every 6 |
| | | | months |
| | | 1 | 1 infiltration every 10 |
| | | | months |
| | | 2.5 | 1 infiltration every 10 |
| | | | months |
| Forehead and | 1₍₃₉₎ | 1 | 1 infiltration |
| periorbicular | | | |
| Forehead and | 1₍₂₉₎ | 1.5 | 2 infiltrations, one every |
| periorbicular | | | 15 days |
| | | 1.8 | 2 infiltrations, one every |
| | | | 30 days |
| | | 2 | 2 infiltrations, one every |
| | | | 15 days |
| | | 4 | 1 infiltration every 30 |
| | | | days |
| | | 6.5 | 1 infiltration every 30 |
| | | | days |
| Forehead and | 1₍₄₀₎ | 2 | Start |
| brow | | 2.5 | 1 infiltration every 30 |
| | | | days |
| Nasogenian and | 1₍₅₀₎ | 2 | 3 infiltrations, one every |
| periorbicular | | | 30 days |
| | | 0.7 | 1 infiltration every 12 |
| | | | months |
| Nasogenian and | 2₍₃₆₎ | 1.5 | 1 infiltration |
| periorbicular | | | |
| Nasogenian and | 3₍₆₁₎ | 2 | Start |
| periorbicular | | 2.5 | 1 infiltration every 4 |
| | | | months |
| | | 3 | 1 infiltration every 5 |
| | | | months |
| Nasogenian and | 4₍₄₅₎ | 2.5 | 4 infiltrations, one every |
| periorbicular | | | 5 months |
| Nasogenian and | 5₍₄₄₎ | 3.5 | Start |
| periorbicular | | 3 | 1 infiltration every 4 |
| | | | months |
| Nasogenian and | 1₍₆₂₎ | 1.03 | 2 infiltrations, one every |
| periorbicular | | | 30 days |
| Nasogenian and | 2₍₄₀₎ | 3 | 1 infiltration |
| periorbicular | | | |
| Nasogenian and | 3₍₂₆₎ | 1 | 6 infiltrations, two every |
| periorbicular | | | 2 months, one every 5 |
| | | | months, one every year, one |
| | | | every 3 months, one every 9 |
| | | | months and one every month |
| Nasogenian and | 4₍₃₆₎ | 2.5* | 3 infiltrations, one every |
| periorbicular | | | 6 months, one at 3 months |
| | | | and one every month |
| Nasogenian and | 5₍₅₀₎ | 3 | Start |
| periorbicular | | 4 | 1 infiltration every 30 |
| | | | days |
| | | 3.5 | 2 infiltrations, one every |
| | | | 15 days |
| | | 3 | 1 infiltration every 15 |
| | | | days |
| Nasogenian and | 6₍₄₈₎ | 2 | 8 infiltrations, one every |
| periorbicular | | | 30 days |
| Nasogenian and | 7₍₆₁₎ | 2.5 | Start |
| periorbicular | | 3 | 1 infiltration every 12 |
| | | | months |
| | | 2 | 2 infiltrations, one every |
| | | | 15 days |
| Nasogenian and | 8₍₅₈₎ | 3 | 2 infiltrations, one every |
| periorbicular | | | 2 months |
| Nasogenian and | 1₍₂₇₎ | 1.5 | 2 infiltrations, one every |
| lateral | | | 30 days |
| commissures | | 0.5 | 1 infiltration at 9 months |
| | | 1 | 1 infiltration at 8 months |
| Nasogenian and | 2₍₅₈₎ | 0.8 | Start |
| lateral | | 1.4 | 1 infiltration every 30 |
| commissures | | | days |
| | | 1.5 | 1 infiltration every 5 |
| | | | months |
| | | 1 | 1 infiltration every 4 |
| | | | months |
| | | 1.3 | 1 infiltration every 15 |
| | | | days |
| | | 1.5 | 1 infiltration every 3 |
| | | | months |
| Nasogenian and | 3₍₅₁₎ | 1 | 2 infiltrations, one every |
| lateral | | | 15 days |
| commissures | | | |
| Nasogenian and | 4₍₃₁₎ | 2 | 4 infiltrations, one every |
| lateral | | | 3 months |
| commissures | | | |
| Nasogenian and | 5₍₃₂₎ | 2 | 2 infiltrations, one every |
| lateral | | | 15 days |
| commissures | | 1 | 1 infiltration every 5 |
| | | | months |
| Nasogenian and | 1₍₃₀₎ | 2 | Start |
| brow | | 1.02 | 6 infiltrations, one every |
| | | | 30 days |
| | | 1 | 4 infiltrations, one every |
| | | | 4 months |
| | | 0.6 | 1 infiltration every 30 |
| | | | days |
| | | 1.2 | 1 infiltration every 2 |
| | | | months |
| Nasogenian and | 2₍₃₇₎ | 2 | Start, and 1 infiltration |
| brow | | | two years after |
| Nasogenian and | 3₍₄₅₎ | 2 | 4 infiltrations, one every |
| brow | | | 45 days |
| Nasogenian and | 4₍₄₁₎ | 1.5 | 2 infiltrations, one every |
| brow | | | 30 days |
| | | 1 | 1 infiltration every 5 |
| | | | months |
| | | 0.7 | 1 infiltration every 30 |
| | | | days |
| | | 0.8 | 2 infiltrations, one every |
| | | | 2 months |
| | | 0.7 | 1 infiltration every 3 |
| | | | months |
| | | 0.6 | 1 infiltration every 6 |
| | | | months |
| | | 0.8 | 1 infiltration every 30 |
| | | | days |
| Nasogenian and | 5₍₅₅₎ | 2 | 1 infiltration every 30 |
| brow | | | days |
| | | 0.5 | 1 infiltration every 30 |
| | | | days |
| | | 1.5 | 1 infiltration every 30 |
| | | | days |
| Nasogenian and | 6₍₃₉₎ | 2.5 | 2 infiltrations, one every |
| brow | | | 15 days |
| Nasogenian and | 7₍₅₆₎ | 1 | 3 infiltrations, one every |
| brow | | | week |
| Nasogenian and | 8₍₃₀₎ | 2 | 2 infiltrations, one every |
| brow | | | 30 days |
| Brow and | 1₍₃₇₎ | 2 | 2 infiltrations, one every |
| periorbicular | | | 30 days |
| Brow, | 1₍₃₇₎ | 2 | 3 infiltrations, one every |
| periorbicular | | | 30 days |
| and nasogenian | | | |
| Brow, | 2₍₅₀₎ | 2.5 | 4 infiltrations, one every |
| periorbicular | | | 30 days |
| and nasogenian | | | |
| Brow, | 3₍₆₀₎ | 1.5 | 3 infiltrations, one every |
| periorbicular | | | 2 months |
| and nasogenian | | 2.5 | 1 infiltration every 12 |
| | | | months |
| Brow, | 4₍₄₇₎ | 2 | 4 infiltrations, one every |
| periorbicular | | | month, 2 every 2 months and |
| and nasogenian | | | one every 4 months |
| Brow, | 5₍₄₇₎ | 2.5 | 2 infiltrations, one every |
| periorbicular | | | 3 months |
| and nasogenian | | | |
| Brow, | 6₍₃₀₎ | 2 | Start |
| periorbicular | | 1 | 1 infiltration every 2 |
| and nasogenian | | | months |
| Brow, | 1₍₃₇₎ | 3.5 | 3 infiltrations, one every |
| periorbicular | | 4 | 3 months and two every 30 |
| and nasogenian | | | days |
| Brow, | 2₍₆₂₎ | 1.2 | 1 infiltration every 30 |
| periorbicular | | | days |
| and nasogenian | | 2 | 3 infiltrations, one every |
| | | | 30 days |
| | | 1.5 | 5 infiltrations, one every |
| | | | 30 days |
| | | 1 | 1 infiltration every 30 |
| | | | days |
| | | 1.2 | 1 infiltration every 2 |
| | | | months |
| Brow, | 3₍₄₇₎ | 3.5 | 6 infiltrations, 4 every 15 |
| periorbicular | | | days, one every 30 days and |
| and nasogenian | | | one every 7 months |
| Brow, | 4₍₃₀₎ | 3 | Start |
| periorbicular | | 3.5 | 1 infiltration every 45 |
| and nasogenian | | | days |
| Brow, | 1₍₃₃₎ | 1 | 2 infiltrations, one every |
| nasogenian and | | | 5 months |
| lateral | | 2 | 1 infiltration every 3 |
| commissures | | | years |
| | | 1 | 3 infiltrations, one every |
| | | | 3 months |
| | | 1.5 | 1 infiltration every 5 |
| | | | months |
| Brow, | 2₍₃₃₎ | 0.5 | Start |
| nasogenian and | | 1 | 4 infiltrations, one every |
| lateral | | | 3 months |
| commissures | | 1.5 | 1 infiltration every 5 |
| | | | months |
| Nasogenian | 1₍₅₈₎ | 2.5 | Start |
| | | 2.8 | 1 infiltration every 15 |
| | | | days |
| | | 3.4 | 1 infiltration every 2 |
| | | | months |
| Nasogenian, | 1₍₃₉₎ | 1.5 | 4 infiltrations, two every |
| periorbicular | | | 30 days and two every 9 |
| and forehead | | | months |
| Nasogenian, | 2₍₄₉₎ | 2.5 | 2 infiltrations, one every |
| periorbicular | | | 30 days |
| and forehead | | | |
| Nasogenian, | 3₍₃₄₎ | 2 | 3 infiltrations, one every |
| periorbicular | | | 30 days |
| and forehead | | | |
| Nasogenian, | 1₍₄₀₎ | 2.3 | 2 infiltrations, one every |
| brow, | | | 30 days |
| periorbicular | | 1.5 | 2 infiltrations every 4 |
| and lateral | | | months |
| commissures | | 2 | 1 infiltration every 5 |
| | | | months |
| Nasogenian, | 2₍₄₈₎ | 0.8 | Start |
| brow, | | 2.5 | 2 infiltrations, one every |
| periorbicular | | | 3 months |
| and lateral | | | |
| commissures | | | |
| Nasogenian, | 1₍₅₁₎ | 2.5 | 2 infiltrations, one every |
| periorbicular, | | | 30 days |
| forehead, | | | |
| brow, lateral | | | |
| commissures | | | |
| Nasogenian, | 2₍₅₀₎ | 3 | Start |
| periorbicular, | | 1.6 | 1 infiltration every 7 |
| forehead, | | | months |
| brow, lateral | | | |
| commissures | | | |
| Nasogenian, | 3₍₄₄₎ | 4 | 3 infiltrations, one every |
| periorbicular, | | | 30 days |
| forehead, | | 3.5 | 1 infiltration every 4 |
| brow, lateral | | | months |
| commissures | | 2.25 | 1 infiltration every 4 |
| | | | months |
| | | 4 | 1 infiltration every 9 |
| | | | months |
| Nasogenian, | 4₍₄₃₎ | 3.5 | Start |
| periorbicular, | | 3 | 1 infiltration every 30 |
| forehead, | | | days |
| brow, lateral | | | |
| commissures | | | |
| Cheek | 1₍₂₉₎ | 3 | 3 infiltrations, one every |
| | | | week |
| | | 2 | 5 infiltrations, one every |
| | | | week |
| | | 2.5 | 7 infiltrations, one every |
| | | | 30 days |
| Scar in | 1₍₅₂₎ | 1.5 | 3 infiltrations, two every |
| periorbital | | | 15 days and one every 3 |
| region | | | months |

AVERAGE OF INFILTRATIONS OF COLLAGEN-
POLYVINYLPYRROLIDONE/PERHYDROSQUALENE IMPLANT

| | Number of | | |
|---|---|---|---|
| Region | patients | Dose (ml) | Frequency |
| Forehead | 1 | 1 | 2 infiltrations, one every |
| | | | 30 days |
| Periorbicular | 1 | 1 | 1 infiltration every 4 months |
| Brow | 5 | 0.4 | 1 infiltration every 30 |
| | | | days |
| Nasogenian | 7 | 1.3 | 2 infiltrations every 30 |
| | | | days |
| Lateral | 2 | 3 | 2 infiltrations, one every |
| commissures | | | 30 days |
| Forehead and | 2 | 1.9 | 1 infiltration every 6 |
| nasogenian | | | months |
| Forehead and | 2 | 2.7 | 2 infiltrations, one every |
| periorbicular | | | 30 days |
| Forehead and | 1 | 2 | 1 infiltration every 30 |
| glabelar | | | days |
| Nasogenian and | 13 | 2.25 | 2 infiltrations every 3 |
| periorbicular | | | months |
| Nasogenian and | 5 | 1 | 1 infiltration every 30 |
| lateral | | | days |
| commissures | | | |
| Nasogenian and | 8 | 1.2 | 2 infiltrations every 2 |
| glabelar | | | months |
| Glabelar and | 1 | 2 | 2 infiltrations, one every |
| periorbicular | | | 30 days |
| Glabelar, | 10 | 2.25 | 2 infiltrations every 3 |
| periorbicular | | | months |
| and nasogenian | | | |
| Glabelar, | 2 | 1 | 2 infiltrations every 3 |
| nasogenian and | | | months |
| lateral | | | |
| commissures | | | |
| Nasogenian, | 3 | 2 | 3 infiltrations, one every |
| periorbicular | | | 30 days |
| and forehead | | | |
| Nasogenian, | 6 | 2.6 | 2 infiltrations every 3 |
| glabelar, | | | months |
| periorbicular | | | |
| and lateral | | | |
| commissures | | | |
| Cheek | 1 | 2.3 | 5 infiltrations, one every |
| | | | week |
| Eyelid scar | 1 | 1.5 | 3 infiltrations, one every |
| | | | month |

1. A composition based on collagen-polyvinylpyrrolidone and perhydrosqualene for filling, through intracutaneous route, minor skin depressions in order to improve its aspect.

2. A composition based on collagen-polyvinylpyrrolidone and perhydrosqualene according to claim 1, characterized because the collagen-polyvinylpyrrolidone is in a citrate solution having a pH ranging from 9.0 to 10.5.

3. A composition based on collagen-polyvinylpyrrolidone and perhydrosqualene according to claim 1, characterized because the perhydrosqualene is a pharmacologically acceptable hydrogenated derivative of animal origin.

4. A composition based on collagen-polyvinylpyrrolidone and perhydrosqualene according to claim 1, characterized because the ratio between the alkaline complex of the collagen-polyvinylpyrrolidone and the perhydrosqualene ranges from 1:5.7 to 1:11.5.

5. A composition based on collagen-polyvinylpyrrolidone and perhydrosqualene according to claim 1, characterized because it is a dermal implantable formula based on perhydrosqualene.

6. A composition based on collagen-polyvinylpyrrolidone and perhydrosqualene according to claim 1, characterized because, owing to the immunomodulating properties of the copolymer, the collagen-polyvinylpyrrolidone favors the acceptation of lipophilic materials by the treated tissue.

7. A composition based on collagen-polyvinylpyrrolidone and perhydrosqualene according to claim 1, characterized because the emulsification can be enriched with polysiloxane.

8. Process for preparing a composition based on collagen-polyvinylpyrrolidone and perhydrosqualene characterized because it comprises the following steps:
(1) forming the collagen-polyvinylpyrrolidone complex;
(2) alkalinizing the collagen-polyvinylpyrrolidone complex, and
(3) forming the final emulsion.

9. Process for preparing the composition based on collagen-polyvinylpyrrolidone and perhydrosqualene, characterized because the collagen-polyvinylpyrrolidone is a biological-synthetic copolymer and because it includes gamma irradiating the mixture of collagen and polyvinylpyrrolidone in an acidic pH.

10. Process for preparing the composition based on collagen-polyvinylpyrrolidone and perhydrosqualene, characterized because the alkalinization of the collagen-polyvinylpyrrolidone complex is conducted until a pH ranging from 9.0 to 10.5 is reached, favoring the integration of the other components when they are added.

11. Process for preparing the composition based on collagen-polyvinylpyrrolidone and perhydrosqualene, characterized because in the step of forming the final emulsification, the alkaline complex of collagen-polyvinylpyrrolidone is mixed with 2,6,10,15,19,23-hexamethyltetracosane to produce a stable, foamless final emulsion.

12. Process for preparing the composition based on collagen-polyvinylpyrrolidone and perhydrosqualene according to claim 10, characterized because the mixture is stabilized with a gelling agent.

13. Process for preparing the composition based on collagen-polyvinylpyrrolidone and perhydrosqualene according to claim 10, characterized because the mixture is stabilized with dibutyl-lauroyl glutamide.

14. Process for preparing the composition based on collagen-polyvinylpyrrolidone and perhydrosqualene according to claim 10, characterized because the mixture can be enriched with polysiloxane.

15. Process for preparing the composition based on collagen-polyvinylpyrrolidone and perhydrosqualene according to claim 1, characterized because the alkaline complex of collagen-polyvinylpyrrolidone to 2,6,10,15,19,23-hexamethyltetracosane ratio is preferably ranging from 1:5.7 to 1:11.5.

16. Use of a composition according to claim 1 for temporarily and safely filling cutaneous depressions.
